(19) United States
(12) Patent Application Publication
(10) Pub. No.: US 2008/0262308 A1
(43) Pub. Date: Oct. 23, 2008

(54) METHOD AND SYSTEM FOR PERFORMING CONTINUOUS FLOW ENDOSCOPY

(75) Inventors: ANNA PRESTEZOG, Sunnyvale, CA (US); JENNIFER HODOR, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Percutaneaus Systems, Inc., Mountain View, CA (US)

(21) Appl. No.: 12/037,311

(22) Filed: Feb. 26, 2008

Related U.S. Application Data
(60) Provisional application No. 60/884,340, filed on Feb. 27, 2007.

(51) Int. Cl. A61B 1/015; A61B 1/307

(52) U.S. Cl. 600/123; 600/156; 600/135; 600/158

(57) ABSTRACT

A fluid irrigation system comprises a small, flexible endoscope and a flexible sheath which is positioned coaxially over the endoscope. A working channel of the endoscope provides a path for introducing an irrigant fluid into a target body lumen or cavity. The irrigant and other fluids may then be removed from the target body lumen or cavity through an axial passage of the sheath. The volume or pressure of the irrigant fluid introduced into the body lumen or cavity is controlled by adjusting the outflow of fluid drained through the sheath.

### CROSS-REFERENCES TO RELATED APPLICATIONS

The present application claims the benefit of provisional U.S. Application No. 60/884,340 (Attorney Docket No. 021807-003700US), filed Feb. 27, 2007, the full disclosure of which is incorporated herein by reference.

### BACKGROUND OF THE INVENTION

1. Field of the Invention

The present invention relates generally to medical apparatus and procedures. More particularly, the present invention relates to the use of small endoscopes for performing diagnostic and therapeutic procedures with continuous introduction and removal of fluids.

The term “endoscopy” refers to the use of a catheter or other tubular device, commonly referred to as an endoscope, for viewing the inside of a body cavity or lumen. Endoscopes usually include fiberoptic bundles for both viewing and providing a light source, and may optionally include one or more “working channels” for permitting the introduction and removal of fluids as well as the introduction of tools for intervention and diagnosis. Some endoscopes have eliminated fiberoptics by placing an imaging chip and/or light source at the distal end of the device.

The size and nature of endoscopes can vary widely. Relatively large diameter endoscopes are used for colonoscopy and gastroscopy procedures where multiple and/or large diameter working channels provide access for a variety of different procedures. In contrast, small diameter, flexible endoscopes used in urological procedures often have only a single, very small diameter working channel which provides limited access for the introduction and removal of fluids as well as for the introduction of working tools. Typically, the working channel exits the distal end of the endoscope in close proximity and parallel to the axis of the imaging lens, thus irrigation fluid passing through the working channel will clarify the fluid volume in front of the lens.

Of particular interest to the present invention, relatively small endoscopes, referred to as flexible cystourethroscopes, are typically used in diagnostic examinations and for short therapeutic procedures in the lower urinary tract, including the bladder. The small size and highly flexible nature and steerability of the flexible endoscope makes it ideal for use in doctor's offices as well as in hospitals for short diagnostic procedures. They have small diameters, typically about 4-6 mm, steerable distal tips for navigating the anatomy of the urethra, and are sufficiently flexible to conform to the anatomy, making them less painful and useable without general anesthesia.

The small size of the working channel of the flexible endoscope, (which allows a compact size of the overall device) however, limits the type and shortens the of procedures that can be performed. Procedures which require the introduction of an irrigant to improve visibility can only be performed until the bladder, urinary lumen or uterus becomes filled with fluid. Excessive fluid pressure caused by over-filling, can cause significant patient discomfort as well as dangerous diffusion of irrigant into the venous bloodstream. Thus, procedures that require more than several minutes must often be interrupted to allow aspiration or drainage of the fluid to empty the bladder before additional fluid can be introduced to continue the procedure. Repeated introduction and removal of the endoscope can result loss of target acquisition and increased trauma to the tissues.

For these reasons, it would be desirable to provide methods and systems which allow for the continuous flow of fluid irrigants through a flexible endoscope or other small endoscope to allow longer procedures to be performed. It would be particularly useful if such continuous flow systems would provide for the flushing and removal of blood, debris, and other substances from the body lumen or cavity which is being observed or treated. Such continuous flow systems will preferably at least substantially preserve the atraumatic characteristics of the small diameter endoscope, preserve the optical performance, working channel and preferably should further preserve the ability to steer the distal end of the endoscope without significant hindrance. At least some of these objectives will be met by the inventions described hereinbelow.

2. Description of the Background Art

U.S. Pat. Nos. 5,989,183; 4,991,565; and 4,974,580, describe disposable endoscope sheaths having auxiliary external fluid channels. U.S. Pat. No. 4,468,216 describes a vacuum catheter which could be inserted into the working channel of an endoscope. U.S. Pat. No. 5,520,636, describes pressure control of an inlet fluid to flush the bladder. Denes et al., (2006) was a poster presentation at the 20th Annual Meeting of Engineering and Urological Society, presenting a study using a urethral introducer sheath (Cystoglide™), Percutaneous Systems, Inc. (PercSys), Mountain View, Calif., the assignee of the present application, for continuous flow cystoscopy. Dr. Denes, the author, is a consultant and advisor to the assignee of the present application.

### BRIEF SUMMARY OF THE INVENTION

The present invention provides methods and systems for the continuous and simultaneous introduction and drainage of irrigation fluid to and from body lumens and cavities. The irrigation fluids will be introduced or removed through a working channel of a small, flexible endoscope and will be removed or introduced through a flow path defined by a flexible sheath disposed coaxially about the endoscope. Introduction and removal of the irrigant fluid will be particularly useful for flushing and removing debris from the visual field of the endoscope to permit continuous viewing without the need to periodically flush and aspirate the viewing field, as has typically been the case with prior small flexible endoscope systems. In some cases, it will be desirable to reverse the flow direction in order to dislodge debris that may have become lodged in the working channel or sheath flow path. The small, flexible endoscopes typically have outer diameters between about 2-7 mm, usually below about 5.5 mm, with room only for the single working channel, which typically has a diameter between about 1-4 mm, typically below about 3.5 mm.

The present invention further provides for the control of volume or pressure within the body lumen or cavity which is receiving the irrigation fluid. The irrigation fluid which is usually introduced through the working channel of the endoscope will typically be allowed to enter at a rate which is determined by the flow resistance of the working channel. The volume of the irrigation fluid which is retained within the body lumen or cavity or the pressure of said fluid within the cavity is controlled by maintaining or adjusting the drainage flow rate of the irrigation fluid through the flexible sheath. In this way, the flow rate of irrigation fluid through the body lumen or cavity can be maximized while controlling distension and preventing excess volume or pressure within the lumen or cavity. While generally less desirable, the irrigation fluid can also be introduced through the sheath and drained through the working channel of the endoscope while controlling volume or pressure as noted.

The systems and methods of the present invention will find particular use for visually guided navigation through the urethra, for introducing and removing irrigation fluid from the bladder, typically in conjunction with imaging the bladder, and for performing medical procedures such as biopsies and fulgarations through the flexible endoscope. The present invention can also be used for viewing and optionally treating the kidney by locating a smaller diameter and longer endoscope and sheath further through the ureter, into the kidney. The methods and systems could be used in a variety of other body lumens and cavities, including the uterus, the stomach, intestine, peritoneum, or sinus tract.

In a first specific aspect of the present invention, methods for irrigating a body lumen or cavity comprise positioning a flexible endoscope in the body lumen or cavity, where the flexible endoscope is positioned in a central passage of a flexible sheath. An irrigation fluid is introduced through a channel in the flexible endoscope into the body lumen. The irrigation fluid is drained through a drainage path defined by the flexible sheath, where the drainage flow rate is controlled to maintain or limit volume or pressure of the irrigation fluid in the body cavity or lumen.

Usually, draining of the irrigation fluid through the drainage path is passive, that is accomplished by gravity and/or as a result of internal pressure within the body lumen or cavity. In other cases, however, the drainage could be enhanced by conventional aspiration techniques, such as pumping, application of a vacuum (for example from an available vacuum source), or the like. In the case of passive drainage, the flow rate will be adjusted by varying a flow resistance in the drainage path, typically by adjusting a valve or variably clamping a drain tube attached to the drainage path. When aspirating with the pump, the drainage flow rate may be maintained by controlling the pumping duration, rate, or some combination thereof. When aspirating using a vacuum, the flow rate may be adjusted by controlling the level or duration of the vacuum. With both pumps and vacuum drainage, however, the flow rate could also be adjusted by adjusting the flow resistance within the drainage path.

The flexible sheath may provide the drainage path in a variety of ways. In one example, at least a portion of the central passage of the sheath is non-collapsible under the conditions of use so that the drainage path is defined between an outside surface of the flexible endoscope and an inside surface of the central passage of the sheath. In addition or alternatively, various surface features could be provided on an inside surface of the flexible sheath in order to maintain spacing between the sheath and the outside surface of the endoscope. Such surface features could also form flow channels, including axial flow channels, helical flow channels, or the like. Still further alternatively, flow channels could be provided between the sheath and the flexible endoscope by an cylindrical insert or other structure having the flow channels defined therein as channels or grooves within its wall or on its outside diameter.

The flexible endoscope and flexible sheath may introduced to the body lumen or cavity in a variety of ways. For example, the flexible endoscope may first be positioned in the body lumen or cavity, with the flexible sheath outside the body then being advanced into the body and/or positioned over the outside of the endoscope. Alternatively, the flexible sheath could first be positioned in the body lumen, and the flexible endoscope then positioned through the central passage of the flexible sheath. Once in position, the flexible endoscope may be connected to a source of irrigation fluid, such as a fluid bag which is raised to a height sufficient to provide gravity flow through the working channel at the desired rate. Alternatively, the irrigation fluid could be delivered by a pump through the working channel, although use of the pump will generally be less desirable. The drainage path defined by the flexible sheath, in turn, will typically be connected to a drain tube which in turn is directly or indirectly connected to a drainage bag or other receptacle. For gravity flow, the drainage bag will be maintained at a level well below the patient, where the drainage flow rate can be controlled by varying the level or the resistance within the drainage tube. Alternatively, the drain tube could be connected to a pump or vacuum source in order to control the drainage rate as discussed above. While the irrigation fluid is being introduced and drained, the target body lumen or cavity can be clearly imaged using the flexible endoscope, despite the presence of blood or debris. Additionally, therapeutic and interventional procedures could be performed through the working channel of the endoscope, although usually placement of instruments in the working channel will cause at least a partial reduction in its flow capacity.

In a second aspect, the present invention provides a system for irrigating a body lumen or cavity comprising a flexible endoscope and a flexible sheath. The flexible endoscope has a single channel for introducing fluids, and the flexible sheath has a distal tip and a central passage for slidably receiving the flexible endoscope. The flexible sheath is further adapted to provide a drainage path for irrigation fluids past the distal end of the sheath. The distal end of the sheath is either blunted to present an atraumatic leading edge, or is tapered to conform to or transition to the outside surface of the flexible endoscope. Usually, the distal tip will have openings to permit entry of the irrigation fluids into the drainage path, where the openings may be on the end of the sheath and/or closely proximal to the end of the sheath. Usually, the sheath will be adapted to drain the irrigation fluid without pumping or applying a vacuum. Alternatively, however, the system could further comprise an aspiration pump or vacuum source which is adapted to connect to the drainage path in order to assist in maintaining fluid drainage therethrough. In all cases, the system could still further comprise a variable flow resistor in the drainage path to control the drainage rate through the drainage path, this will also determine the flow through the entire system as long as the unrestricted drainage flow capacity of the sheath exceeds the inflow capacity through the endoscope.

The systems will typically further include a source of irrigation fluid adapted to connect to the single channel of the flexible endoscope. The irrigation fluid source will typically be adapted to deliver the fluid to the central passage via a gravity flow. In other instances, however, a pump could be provided to deliver the irrigation fluid to the central passage. In still further instances, the systems of the present invention could comprise a controller which monitors irrigation volume and drainage volume to control the amount or pressure of irrigation fluid in the body lumen or cavity An optional pressure relief valve on the drain which allows fluid to drain when a predetermined pressure in the body lumen is exceeded, would allow distension of the organ to be maintained throughout the procedure. This would limit or eliminate the flow rate manipulations required by the user to accomplish the same results.

In a third aspect, the present invention provides flexible sheaths which comprise a sheath body having a proximal end, a distal end, and a central passage therethrough. The distal end is typically tapered to conform to the flexible endoscope, and the central passage is dimensioned to provide a fluid drainage path when the flexible endoscope is present in the central passage. Usually, the drainage path will have a significantly greater flow capacity than that of the endoscope channel. Optionally, an inner surface of the central passage may have surface features which help define the drainage path between the flexible endoscope and said inner wall. The surface features may be formed continuously over at least most of the length of the inner wall, typically being axial ribs, helical ribs, serpentine ribs, or the like. Alternatively, the surface features could be formed discontinuously and distributed over at least most of the wall, for example in the form of knobs, protrusions, bumps, or other radial spacers. Thirdly, the surface features could be inflatable, which would be particularly advantageous with a highly flexible sheath wall that presents a small cross sectional profile during advancement and which could then be inflated to provide sufficient rigidity and space for defining the drainage path.

In the exemplary embodiments, the sheath body has at least one opening which permits fluid drainage therethrough when the flexible endoscope is present in the central passage. The sheath body will typically also include a proximal hub at its proximal end, where the hub includes an axial port for receiving the flexible endoscope and at least one additional port for connecting the drain path for exiting fluids. Usually, for males, the sheath body will have length in the range from 10 cm to 30 cm, an outer diameter in the range from 7 mm to 9 mm, and an inner central passage diameter in the range from 5 mm to 7 mm. For females, the length may be shorter down to 3 cm while the width may be wider by up to 0.5 cm.

### BRIEF DESCRIPTION OF THE DRAWINGS

FIG. 1 illustrates a system for irrigating a body lumen or cavity constructed in accordance with the principles of the present invention.

FIG. 1A illustrates a variable resistance flow element comprising an adjustable clamp located in a drainage tube of the system of FIG. 1.

FIG. 2 illustrates a flexible sheath having a central passage for receiving a flexible endoscope (shown in broken line) in accordance with the principles of the present invention.

FIG. 3 is a detailed view of the distal end of the flexible sheath and flexible endoscope of FIG. 2, shown with the distal end of the endoscope in both a straight (full line) and deflected (broken line) configurations.

FIGS. 4A-4C are alternative cross-sectional views of the flexible sheath and flexible endoscope taken along line 4-4 of FIG. 3.

FIGS. 5 and 6 illustrate further alternative cross-sectional configurations of flexible sheaths having non-circular geometries received over flexible endoscopes.

FIG. 7 is a partial view of a distal end of a flexible sheath and flexible endoscope system, where the flexible sheath includes both an inner insert and outer sheath cover which is retractable relative to the inner insert.

FIG. 8 is a partial view of the distal end of a flexible sheath useful in the systems of the present invention, where the flexible sheath has an expansible distal tip.

FIGS. 9A-9B illustrate a flexible sheath having inflatable surface features in accordance with the present invention.

FIGS. 10A-10D illustrate introduction of the irrigating system of the present invention through a urethra into a bladder and subsequent circulation of irrigating fluid in the bladder using the system.

FIGS. 11A-11C illustrate an exemplary technique for introducing the endoscope and sheath into the urethra.

### DETAILED DESCRIPTION OF THE INVENTION

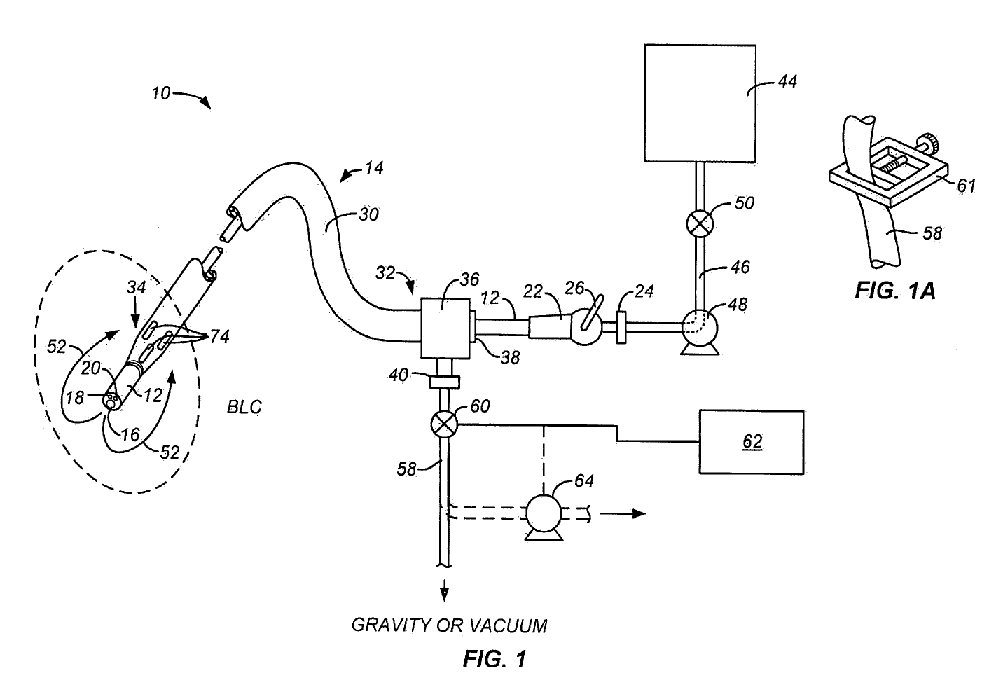

As shown in FIG. 1, a system 10 constructed in accordance with the principles of the present invention comprises a flexible endoscope 12 and a flexible sheath 14. A flexible endoscope 12 will usually be a small diameter scope comprising a flexible polymeric body and having a working channel 16, an illuminating optical fiber bundle 18, and an imaging optical fiber bundle 20 extending therethrough. The endoscope 12 will typically have a diameter below about 7 mm, typically below about 5 mm, with a working channel having a diameter between 1-3 mm, typically below about 2.5 mm. The endoscope length may vary in the range from 25 cm to 50 cm, typically being in the range from 30 cm to 40 cm. The endoscope 12 will typically have a proximal hub 22 which includes a fitting 24 which opens to the working channel 16 and a lever 26 for deflecting a distal region of the endoscope. The systems of the present invention may conveniently employ commercially available endoscopes, such as ACMI ACN-2, Olympus CYS-05 and Storz_{—}11272 Cystourethroscopes.

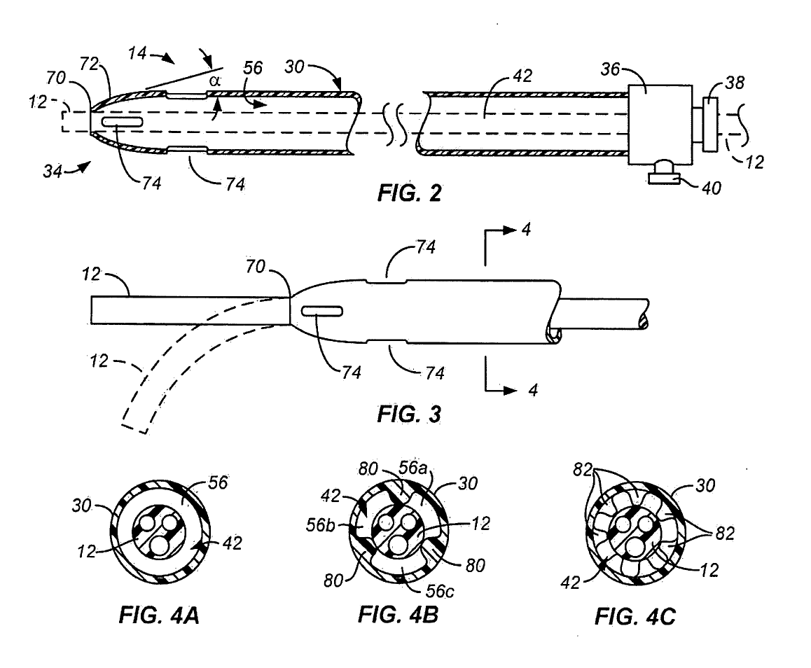

The flexible sheath 14 comprises a sheath body 30 which is typically formed from a polymer, such as a polyolefin, nylon or polyurethane. Conveniently, the polymer may be extruded into a desired tubular configuration having at least one central passage extending from a proximal end 32 to a distal end 34. The flexible sheath 14 also includes a proximal housing 36 at its distal end. The proximal housing 36 includes both an axial port 38 which receives the endoscope 12 and a drainage port 40 which receives fluid from the central passage 42 (FIG. 2) of the sheath. The sheath surfaces in contact with the endoscope and patient tissues preferably have a low friction coating or are designed to be used with applied lubrication.

In use, irrigation fluid may be fed from a source, such as bag 44 through a feed tube 46 which is connected to the fitting 24 on the flexible endoscope 12. Usually, the feed will be by gravity where the bag 44 is elevated from an appropriate support (not shown). Alternatively, a feed pump 48 may be provided to assist in the introduction of irrigation fluid into the endoscope. Usually, a valve 50 or external clamp will be provided in order to control the rate of irrigant flow from the bag, either directly into the endoscope 12 or into the pump 48. Thus, the valve 50 or clamp may be used both to control the flow rate and to shut the flow on and off. Alternatively or additionally, the flow rate may be controlled by varying the speed or other output characteristics of pump 48, when a pump is used.

The irrigation fluid entering from bag 44 through feed tube 46 will flow through the working channel 16 and into a target body lumen or cavity BLC shown in broken line in FIG. 1. The irrigant fluid will flow outwardly from the distal end of the working channel 16 as shown by arrows 52 first coaxially with the axis of the terminal optical bundle, clearing the fluid space in front of the lens, and then eventually exiting into the annular lumen(s) 56 (FIG. 2) of the flexible sheath 30. From the central passage 42, the irrigation fluid will be drained through drainage port 40 into a drain tube 58, where the flow may be induced by gravity, attachment to a vacuum source, pumping, or some combination thereof. As shown in FIG. 1, a valve or clamp 60 is provided to control the drainage rate in order to maintain a desired or target volume of the irrigation fluid in the body lumen or cavity. An external clamping member, such as adjustable clamp 61, maybe mounted on tube 58, as illustrated in FIG. 1A. Such control may be effected based on the observations of the treating physician. Alternatively, the pressure or volume within the body lumen or cavity may be monitored using appropriate sensing equipment. In such cases, a controller 62 may be provided to receive the monitoring information and to control the output flow rate, for example by controlling an automatic valve 60 as shown in full line in FIG. 1. Alternatively, controller 62 could vary the output of an optional pump 64, as shown in broken line in FIG. 1.

Referring now to FIGS. 2, 3, and 4A, the structure of the flexible sheath 14 will be described in more detail. The sheath body 30 is shown in cross-section with the flexible endoscope 12 shown in broken line. The distal end 34 of the sheath body 30 is typically tapered so that it conforms to the body of the flexible endoscope 12 at its distal tip 70 and has a radiused shoulder or conical region 72 extending proximally from the distal tip 70. The length of the conical region will generally be in the range from 0.5 cm to 2 cm. The portions of the sheath body 30 which are proximal to the conical distal portion are generally cylindrical with a constant diameter, although in some instances the diameter could vary and in other instances the shape could be other than circular. Additionally, the tapered surface 72 need not be a true cone, but could also have a generally bullet-shape with a certain degree of curvature. The conical surface 72 will serve as a transition between the smaller diameter distal tip 70 and the larger diameter remainder of the sheath to facilitate advancement of the sheath over the endoscope 12 and/or advancement of the assembly of the sheath and endoscope through a body lumen.

In the exemplary embodiments, one or more drainage ports 74 will be formed near the distal end 34 of the sheath body 30 in order to permit the inflow of irrigant and other fluids from the body cavity or lumen into the central passage 42 of the sheath. The drainage ports 74 may be formed in the tapered region 72 or alternatively in the cylindrical region of the sheath body which is immediately proximal to the tapered region. When the endoscope 12 is present in the central passage 42 of the sheath body 30 as illustrated in FIG. 4A, the annular lumen 56 will be formed. The annular lumen 56 is thus available for the drainage of irrigant or other fluids passing into the passage 42 through the distal drainage ports 74.

Referring now to FIG. 4B, surface features 80 may be formed on the inside surface of the central passage 42 of the sheath body 30. The surface features may be axial ribs which define three separate drainage paths 56A, 56B, and 56C. Alternatively, the surface features may have a helical, serpentine, or other configuration to provide one or more non-linear drainage paths.

As a further alternative, shown in FIG. 4C, the surface features may comprise a plurality of individual bumps or protuberances 82 which are distributed over the inside surface of the sheath body 30. The protuberances 82 will serve to position the endoscope 12 generally centrally through the central passage 42 of the sheath. A variety of other surface features could also be adapted for both centering the endoscope and/or for forming multiple discrete drainage paths within the sheath.

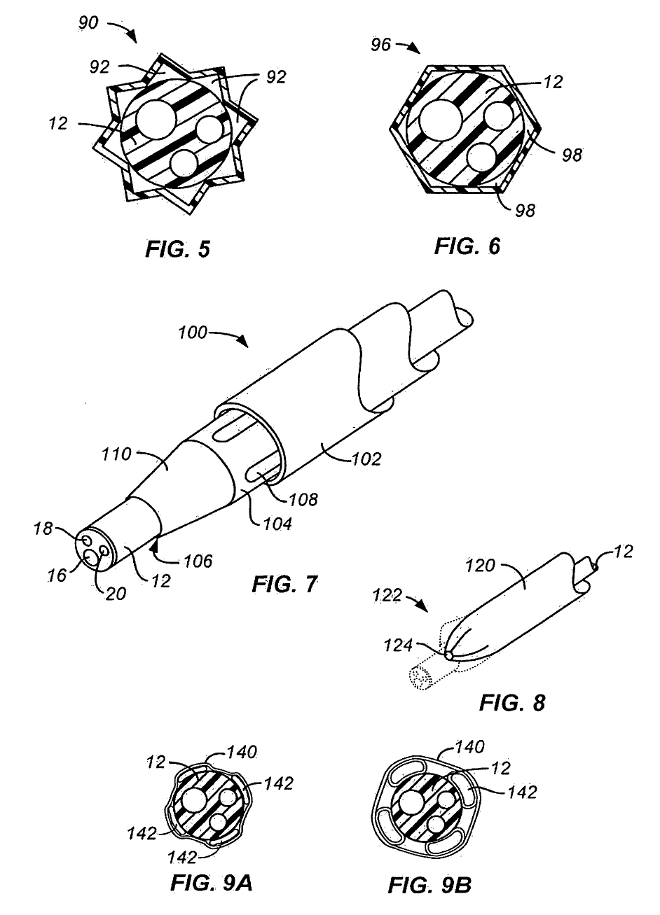

Referring now to FIGS. 5 and 6, the drainage paths provided by the sheath may be formed by using sheaths having non-circular cross-sections. For example, as shown in FIG. 5, a sheath having an eight-sited star configuration can be configured to conform to the outer surface of the endoscope 12. The sheath 90 will thus define a plurality of discrete triangular flow paths or lumens 92, each of which can independently provide for the flow of irrigant from the distal end of the sheath to the proximal end. FIG. 6 shows how a sheath 96 having a hexagonal cross-section can conform to the outer surface of the endoscope 12 and provide six discrete, relatively small flow channels 98. Both the star-shaped sheath 90 and the hexagonal sheath 96 could be formed out of larger widths to provide even more cross-sectional area for allowing the drainage of irrigant and other fluids.

Referring now to FIG. 7, the flexible sheath of the present invention can be formed from two or more components. For example, sheath 100 comprises an outer, generally cylindrical sheath cover 102 and an inner insert 104. The insert has a central lumen 106 which receives the flexible endoscope 12. On its outer surface, the insert 104 has a plurality of axial channels 108 which provide for the desired flow paths. The channels 108, however, can be opened and closed by axially advancing and retracting the outer sheath cover 102. In addition, the drainage rate through the channels can also be adjusted by retracting and advancing the sheath cover. Usually, the insert 104 will have a conical or other tapered surface 110 to provide for the desired transition region which facilitates advancement of the sheath over the endoscope and/or through the body lumen or cavity.

Referring now to FIG. 8, a further embodiment of the flexible sheath 120 can be adapted to have a closed, tapered distal end 122. The tapering can terminate at a distal point 124 which may be entirely closed or have a slightly open end. The sheath is suitable for atraumatic advancement through the target body lumen or cavity. The sheath end 122 can be opened by advancing the flexible endoscope through the end (as shown in broken line) to open the end to allow the inflow of irrigant or other fluids to be drained.

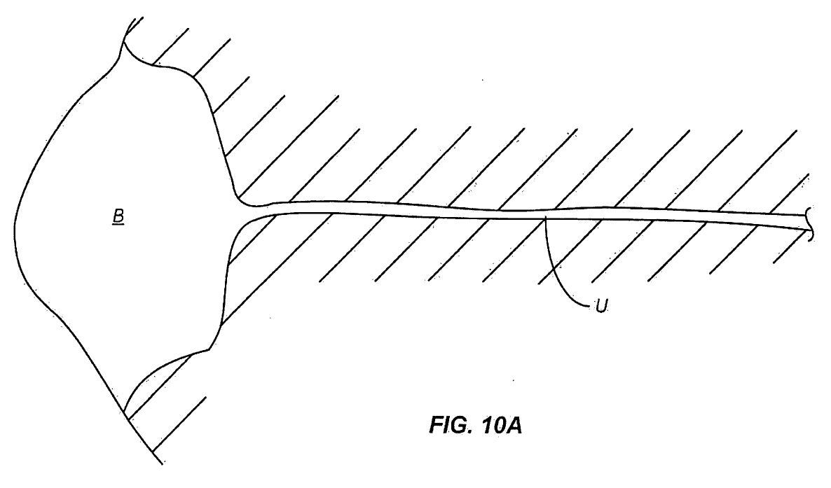

Referring now to FIGS. 9A-9B, further embodiments of the sheath 140 can be constructed to have very thin, highly flexible bodies, typically formed from a thin film or membrane of material. Such sheaths can have inflatable surface structures, such as ribs 142, which can be expanded and rigidified by inflation with a suitable medium, typically saline or other incompressible fluid. Such thin-walled sheaths 140 will typically be delivered over the endoscope 12 or over another insert or relatively stiff introducer member to place them into and through the target body lumen or cavity prior to inflation.

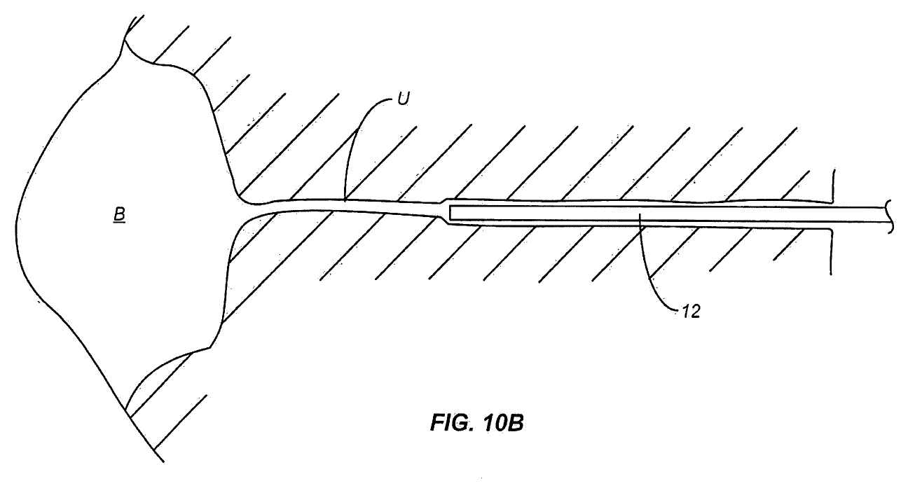

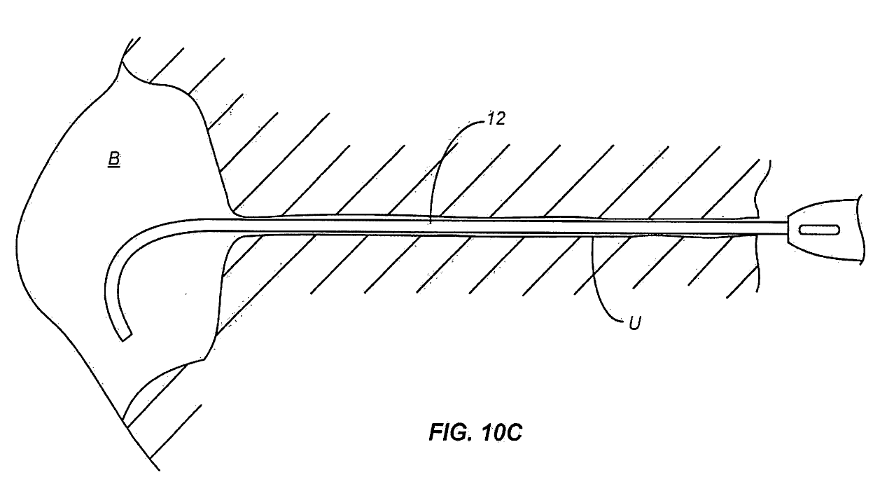

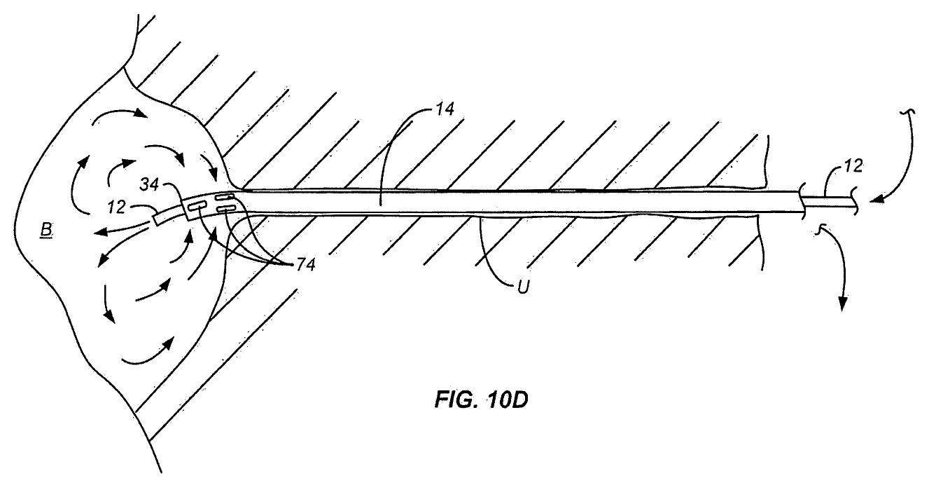

Referring now to FIGS. 10A-10D, introduction of the flexible endoscope and sheath system 10 through a urethra U into a bladder B will be described. The anatomy showing the bladder B at the remote end of the urethra U is shown in FIG. 10A. The endoscope 12 may first be introduced through the urethra U, as shown in FIG. 10B, until it reaches the bladder B, as shown in FIG. 10C. Typically, the distal end of the flexible endoscope 12 may be deflected in order to view different portions of the bladder. In order to introduce an irrigant fluid to clear blood and other debris from the bladder, the flexible sheath 14 may be advanced over the endoscope 12 until the distal end 34 enters the bladder B, as shown in FIG. 10D. The irrigation fluid is then introduced through the working channel of the endoscope 12 so that it flows outwardly into the bladder where it can circulate until it is drained through the ports 74 of the flexible sheath 14. The drainage may be by gravity flow or may be assisted by a pump or vacuum source, as described above in connection with FIG. 1. The relative position of the flexible endoscope 12 and the distal end 34 of the sheath 14 may also be changed while the bladder is being flushed with an irrigant fluid in order to reposition the endoscope for further imaging or for any other purpose. It will be appreciated that imaging may continue while the interior of the bladder is being flushed in order to improve the field of view by removing blood, debris, and other materials which might interfere with the imaging. In order to avoid over pressuring or over extending the bladder, the flow rate of irrigant and other fluids being withdrawn from the bladder through the sheath 14 will be controlled by any of the mechanisms described above in connection with FIG. 1.

While the above is a complete description of the preferred embodiments of the invention, various alternatives, modifications, and equivalents may be used. Therefore, the above description should not be taken as limiting the scope of the invention which is defined by the appended claims.

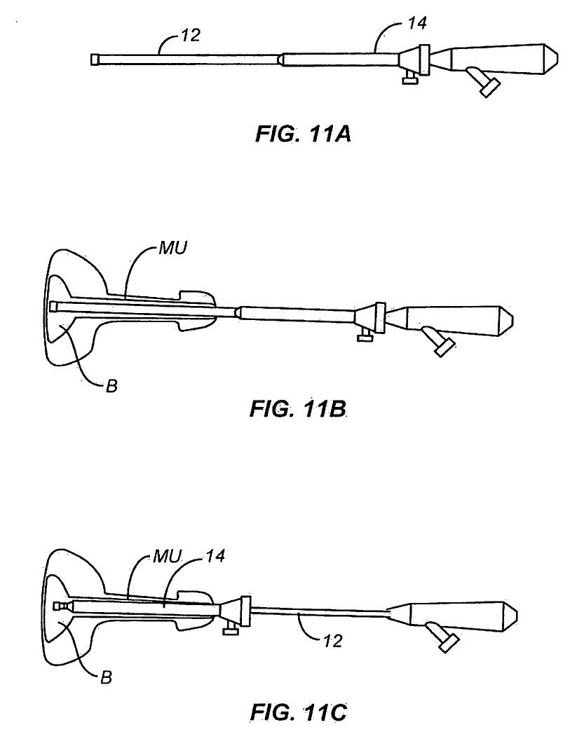

FIGS. 11A-11C illustrate one technique for introducing the endoscope 12 and sheath 14 in the male urethra MU. The sheath 14 is first placed and retracted proximally over the endoscope 12, as shown in FIG. 11A. A distal end of the endoscope 12 is then advanced through the male urethra MU and into the bladder B, as shown in FIG. 11B. After the endoscope 12 has been advanced, the sheath 14 is advanced over the endoscope 12 and into the urethra U so that a distal end reaches the bladder B, as shown in FIG. 11C.

What is claimed is:

1 A method for irrigating a body lumen or cavity, said method comprising:
positioning a flexible endoscope having at least one channel in the body lumen or cavity, wherein the flexible endoscope is positioned in a central passage of a flexible sheath;
introducing an irrigation fluid into the body lumen or cavity; and
draining the irrigation fluid through a drainage path and at a drainage flow rate is maintained to control a volume or pressure of irrigation fluid in the body cavity or lumen, wherein the fluid is introduced or drained through the at least one channel of the flexible endoscope.

2. A method as in claim 1, wherein the fluid is introduced through the channel in the endoscope and drained through a passage in the flexible sheath or between the flexible sheath and an outer surface of the endoscope.

3. A method as in claim 1, wherein the fluid is introduced through a passage in the flexible sheath or between the flexible sheath and an outer surface of the endoscope and drained through the channel in the endoscope.

4. A method as in claim 1, wherein draining is passive without use of pumping or a vacuum, wherein the drainage flow rate is maintained by controlling a flow resistance in the drainage path.

5. A method as in claim 4, wherein the flow resistance is adjusted by adjusting a valve to the drainage path of the flexible sheath.

6. A method as in claim 4, wherein the flow resistance is adjusted by variably clamping a drain tube.

7. A method as in claim 1, wherein the flow rate is adjusted by a pressure relief valve in the drainage path which opens when pressure exceeds a threshold level.

8. A method as in claim 1, wherein draining comprises aspirating or siphoning the irrigation fluid through the drainage path. Aspiration may include extraction by siphon effect.

9. A method as in claim 8, wherein aspirating comprises pumping the irrigation fluid and the drainage flow rate is maintained by controlling the pumping duration or flow rate.

10. A method as in claim 8, wherein aspirating comprises pumping the irrigation fluid and the drainage flow rate is adjusted by controlling a flow resistance in the drainage path.

11. A method as in claim 8, wherein aspirating comprises applying a vacuum from a vacuum source to the drainage path, wherein the drainage flow rate is adjusted by controlling the level or duration of the vacuum.

12. A method as in claim 8, wherein aspirating comprises applying a vacuum from a vacuum source to the drainage path, wherein the drainage flow rate is adjusted by controlling a flow resistance in the drainage path.

13. A method as in claim 1, wherein the body lumen or cavity is selected from the group consisting of the uterus, stomach, esophagus, colon, sinus, intestine bronchi.

14. A method as in claim 13, wherein the body lumen or cavity is a bladder.

15. A method as in claim 1, wherein at least a portion of the central passage is non-collapsible under conditions of use and a fluid flow path is defined between an outside surface of the flexible endoscope and an inside surface of the central passage of the sheath.

16. A method as in claim 1, wherein a fluid flow path is provided by flow channels formed in the flexible sheath.

17. A method as in claim 16, wherein the flow channels are defined by one or more flow channels in an insert in a central passage of the sheath.

18. A method as in claim 1, wherein the flexible endoscope is first positioned in the body lumen or cavity and the flexible sheath is then positioned over the flexible endoscope.

19. A method as in claim 1, wherein the flexible sheath is first positioned in the body lumen and the flexible endoscope is then positioned through the central passage of the flexible sheath.

20. A method as in claim 2, wherein the irrigation fluid is delivered at a rate which is limited by the flow resistance in the channel of the flexible endoscope.

21. A method as in claim 20, wherein the irrigation fluid is delivered by gravity from a fluid bag.

22. A method as in claim 20, wherein the irrigation fluid is delivered by a pump.

23. A method as in claim 20, wherein the irrigation fluid is delivered at a rate in the range from 20 ml/min to 150 ml/min.

24. A method as in claim 1, wherein drainage flow rate is adjusted to maintain a substantially constant volume or pressure of irrigation fluid in the body lumen or cavity.

25. A method as in claim 24, wherein the drainage rate is adjusted by controlling a flow resistance in the drainage path.

26. A method as in claim 25, wherein the resistance is adjusted by a valve or roller clamp.

27. A method as in claim 24, wherein the drainage rate is adjusted by controlling the exit height of a drainage tube relative to the sheath.

28. A method as in claim 1, further comprising imaging the body lumen or cavity through the flexible endoscope.

29. A method as in claim 28, wherein the irrigation fluid is being continuously introduced and drained during imaging.

30. A method as in claim 28, wherein the irrigation fluid is being intermittently introduced and drained during imaging.

31. A method as in claim 28, wherein imaging is performed after the body lumen or cavity has been irrigated to clear the imaging field, wherein irrigation is stopped during at least a portion of the imaging.

32. A system for irrigating a body lumen or cavity, said system comprising:
a flexible endoscope having a single channel for introducing or draining fluids; and
a flexible sheath having a distal tip and a central passage for slidably receiving the flexible endoscope, wherein the flexible sheath is adapted to provide a fluid flow path and the distal tip is tapered to conform to an outside surface of the flexible endoscope.

33. A system as in claim 32, wherein the distal tip has openings to permit passage of irrigation fluids into the drainage path.

34. A method as in claim 32, wherein the sheath is adapted to drain the irrigation fluid without pumping or applying a vacuum.

35. A system as in claim 32, further comprising an aspiration pump or vacuum source which is adapted to connect to the sheath or endoscope to maintain a fluid drainage rate therethrough.

36. A system as in claim 32, further comprising a variable flow restrictor in the drainage path to control drainage rate through the drainage path.

37. A system as in claim 36, wherein the variable flow restriction comprises an external clamping member on a drain tube which provides the drainage path.

38. A system as in claim 32, further comprising a source of irrigation fluid adapted to connect to the single channel of the flexible endoscope or the fluid flow path of the sheath.

39. A system as in claim 38, wherein the irrigation fluid source is adapted to deliver the irrigation fluid to the single channel or central passage via a gravity flow.

40. A system as in claim 38, further comprising a pump to deliver irrigation fluid to the single channel or central passage.

41. A system as in claim 32, further comprising a controller which monitors irrigation volume and drainage volume to control the amount of irrigation fluid in the body lumen or cavity.

42. A system as in claim 32, further comprising a controller which monitors and controls volume or pressure of the irrigation fluid in the body lumen or cavity.

43. A system as in claim 32, wherein the sheath comprises:
a sheath body having a proximal end, a distal end, and a central passage therethrough;
wherein the distal end is tapered to conform to the flexible endoscope and the central passage is dimensioned to provide the fluid flow path when said flexible endoscope is present in the central passage.

44. A system as in claim 43, wherein the flow path has a flow capacity which is greater than that of the endoscope channel.

45. A system as in claim 43, wherein an inner wall of the central passage and/or an insert received in the central passage has surface features which define the drainage path between the flexible endoscope and said inner wall.

46. A system as in claim 45, wherein the surface features are continuous over at least most of the length of the inner wall.

47. A system as in claim 45, wherein the surface features are selected from the group consisting of axial rib(s), helical rib(s), and serpentine rib(s).

48. A system as in claim 45, wherein the surface features are discontinuous and distributed over at least most of the inner wall.

49. A system as in claim 45, wherein the surface features are inflatable.

50. A system as in claim 43, wherein the distal end of the sheath body has at least one opening which permits fluid flow therethrough when the flexible endoscope is present in the central passage.

51. A system as in claim 43, further comprising a proximal hub having an axial port for receiving the flexible endoscope and another port for passing fluids to and from the flow path.

52. A system as in claim 43, wherein the sheath body has a length in the range from 10 cm to 30 cm, an outer width in the range from 7 mm to 9 mm, and an inner central passage diameter in the range from 5 mm to 7 mm.

53. A sheath for use with a flexible endoscope having a single channel for introducing or draining fluids, said sheath comprising:
an elongate sheath body having a proximal end, a distal end, and a central passage therethrough, wherein the central passage is adapted to removably receive the flexible endoscope and to define a fluid flow path, wherein the distal end of the sheath structure has a tapered region to provide a smooth transition to the endoscope when said endoscope is received in the central passage; and
a hub attached to the proximal end of the sheath body, said hub having an axial port for receiving and sealing against the flexible endoscope and a fluid port coupled to the fluid flow path.

54. A sheath as in claim 53, wherein the elongate sheath body comprises a continuous tubular structure with a single central passage.

55. A sheath as in claim 54, wherein at least a portion of the sheath body is non-collapsible when present in a body lumen.

56. A sheath as in claim 55, wherein the tubular structure has a circular cross-section.

57. A sheath as in claim 55, wherein the tubular structure has a non-circular cross-section.

58. A sheath as in claim 53, wherein the distal end has openings to permit flow of fluids to or from the flow path.

59. A sheath as in claim 58, wherein at least some of said openings are disposed in the tapered region.

60. A sheath as in claim 58, wherein at least some of said openings are disposed proximally of the tapered region.

61. A sheath as in claim 55, wherein the central passage of the sheath body has a cross-sectional shape which does not conform to the outer cross-sectional shape of the flexible endoscope so that the drainage path is provided by the interstices between the central passage and the endoscope.

62. A sheath as in claim 61, wherein the sheath body has a polygonal cross-section.

63. A sheath as in claim 53, wherein surface features are disposed over the inner wall of the central passage of the sheath body and/or insert received in the central passage.

64. A sheath as in claim 63, wherein the surface features are continuous over at least most of the length of the sheath body and selected from the group consisting of axial rib(s), helical rib(s), and serpentine rib(s).

65. A sheath as in claim 63, wherein the surface features are discontinuous and distributed over at least most of the inner wall of the central passage.

66. A sheath as in claim 63, wherein the surface features are inflatable.

67. A sheath as in claim 66, wherein the sheath body is radially collapsible so that it may be introduced in a radially collapsible configuration and rigidified by inflating the surface features in situ.

68. A sheath as in claim 53, wherein the central passage of the sheath body terminates in a closed end or small aperture which can be opened by axial advancement of the endoscope therethrough.

69. A sheath as in claim 53, further comprising an insert which is removably received in the central passage and which has a central passage for removably receiving the flexible endoscope, wherein the drainage path is disposed between an outer surface of the insert and an inner surface of the central passage of the elongate sheath body.

70. A sheath as in claim 69, wherein the outer surface of the insert has at least one channel formed therein.

71. A sheath as in claim 69, wherein the central passage of the sheath and the outer surface of the insert have non-conforming cross-sections to provide one or more channels therebetween.

72. A sheath as in claim 69, wherein the channel(s) of the insert are disposed so that distal end(s) of said channels are disposed by axially retracting the sheath body relative to the insert.

73. A sheath as in claim 53, wherein the sheath body has a length in the range from 10 cm to 30 cm, an outer diameter in the range from 7 mm to 9 mm, and an inner central passage diameter in the range from 5 mm to 7 mm.

74. A sheath as in claim 53, wherein at least one of an outer surface and the central passage is at least partially coated with a lubricous material.
